(19) United States
(12) Patent Application Publication
(10) Pub. No.: US 2022/0054286 A1
(43) Pub. Date: Feb. 24, 2022

(54) Stent and Catheter Systems for Treatment of Unstable Plaque and Cerebral Aneurysm

(71) Applicant: MG Stroke Analytics Inc., Calgary (CA)

(72) Inventor: Mayank Goyal, Calgary (CA)

(21) Appl. No.: 17/298,914

(22) PCT Filed: Nov. 5, 2020
(86) PCT No.: PCT/CA2020/051501

§ 371 Date: Jun. 1, 2021

Related U.S. Application Data
(60) Provisional application No. 62/931,614, filed on Nov. 6, 2019.

(51) Int. Cl. A61F 2/852; A61F 2/958; A61L 31/06; A61F 2/966; A61B 17/12; A61F 2/90

(52) U.S. Cl. CPC A61F 2/852; A61F 2002/823; A61L 31/06; A61F 2/966; A61B 17/12136; A61B 17/12118; A61B 17/1204; A61F 2/90; A61F 2210/0004; A61F 2250/0039; A61F 2250/0037; A61F 2250/0018; A61F 2250/0023; A61F 2250/0067; A61F 2/958

(57) ABSTRACT

The invention generally relates to co-axial stent and catheter systems and medical procedures utilizing these systems. The co-axial stent system is characterized by two-coaxial stents, including an outer resorbable stent and an inner metal stent used to effect deployment of the resorbable stent. The stents may use for treatment of unstable plaque and/or thrombus at the carotid bifurcation and particularly those that are not causing any significant stenosis. The stents may also be used for treatment of cerebral aneurysms. The invention further describes related, equipment, uses and kits for the treatment of unstable plaque and/or thrombus and/or aneurysms.

### RELATED APPLICATIONS

This application is related to U.S. provisional application 62/846,467 filed May 10, 2019 and U.S. patent application Ser. No. 16/239,296 filed Jan. 3, 2019, both incorporated herein by reference.

### FIELD OF THE INVENTION

The invention generally relates to co-axial stent and catheter systems and medical procedures utilizing these systems. The co-axial stent system is characterized by two-coaxial stents, including an outer resorbable stent and an inner metal stent used to effect deployment of the resorbable stent. The stents may be used for treatment of unstable plaque and/or thrombus at the carotid bifurcation and particularly those that are not causing any significant stenosis. The stents may also be used for treatment of cerebral aneurysms. The invention further describes related, equipment, uses and kits for the treatment of unstable plaque and/or thrombus and/or aneurysms.

### BACKGROUND OF THE INVENTION

### 1. Introduction

Acute ischemic stroke (AS) or Transient Ischemic Attack (TIA) are acute diseases where tissue death (infarction) may occur in the brain if timely treatment is not applied.

A common cause of AS/TIA is when an emboli breaks free from a development site (typically within the arterial system), which then travels into brain blood vessels. Emboli may have a variety of morphological and/or compositional characteristics, such as being predominantly fatty tissues (atherosclerosis plaque) and/or a blood clot (thrombus).

Atherosclerosis plaques and/or thrombi may form in a number of locations in the body from a variety of triggering factors. One common sources of emboli causing AS/TIA is plaque and/or thrombus that forms at the common carotid artery (CCA) bifurcation where the CCA branches into the internal carotid artery (ICA) and the external carotid artery (ECA).

As atherosclerotic plaque grows within an artery, it will increasingly cause a narrowing or stenosis of the artery and hence a restriction to blood flow. As stenosis increases, a patient may become symptomatic as the decreased blood supply affects tissues distal to the obstruction. In addition, emboli may break off the plaque. Generally, symptoms caused by a narrowing of a vessel will not present until a vessel is more than 50% obstructed. In this case, if a patient becomes symptomatic due to stenosis (for example the patient experiencing sudden weakness) and is not showing symptoms of acute stroke (for example, loss of neurological functions), a number of treatment options are available as will be described below.

In situations where an emboli has broken free and the patient is showing signs of AS/TIA, the severity of symptoms, diagnosis of the location of the resting place of the emboli and/or the origin of the emboli may all contribute to a treatment option decision. For example, one common signal of a significant AS/TIA is amaurosis fugax which presents as a transient loss of vision in the ipsilateral eye. In this case, an emboli may have had origins within the common carotid artery (CCA) and specifically at the CCA bifurcation.

### 2. Unstable Plaque

Importantly, there are also situations where stenosis of an artery such as the CCA and/or the origin of the ICA, is less than 50% and the patient has had or is exhibiting stroke symptoms. Generally, in these cases, symptoms have presented not necessarily because of the blood flow restriction but due to emboli breaking free from the atherosclerotic plaque/thrombus which may then present various neurological symptoms.

These types of plaque/thrombus are referred to as unstable plaque/thrombus insomuch as they are characterized as plaque/thrombus where stenosis is less than 50% and where the patient is exhibiting symptoms.

For reference, FIG. 1 is a schematic diagram of a CCA bifurcation 100. The CCA bifurcation 100 includes a CCA 102, an ICA 104 and an ECA 106. A direction of blood flow 101 shows the normal direction of flow from the CCA 102 to both the ICA 104 and the ECA 106. Exemplary plaque deposits 108a, 108b and 108c are shown at locations where plaque could be deposited proximal to the CCA bifurcation 100. Plaque deposit 108a is located in the ICA 104 and extends annularly around the ICA. Plaque deposit 108b is located on a portion of the ECA 106. Plaque deposit 108c is located on a portion of the CCA 102. For the purposes of description, as an unstable plaque can be varying degrees of atherosclerotic tissue or thrombus and the proportions cannot be readily diagnosed or quantified, this description will refer to unstable plaque with the understanding that an unstable plaque may be comprised of varying proportions of atherosclerotic and thrombus material.

Furthermore, for the purposes of background description, it is important to note that blood supply to the brain is somewhat unique due in part due to the connection between ICAs on both sides of the body through the Circle of Willis. FIG. 2 is a schematic diagram of a Circle of Willis showing a left ICA and a right ICA, which are connected through two pathways: one comprising left and right anterior cerebral arteries and the anterior communicating artery, and the other comprising left and right posterior communicating arteries and left and right posterior cerebral arteries. As such, if blood flow is cut off to one CCA (the ipsilateral side), blood flow may still be maintained to the ipsilateral ICA through the Circle of Willis. The ECA also includes various cross connections where, in the event of occlusion of one ECA (e.g. at the CCA bifurcation; ipsilateral side), the cross connections can provide blood flow to the distal ipsilateral vessels. As is known, there are a number of anatomical variations between individuals that can provide a variety of cross connection patterns.

A variety of treatments are known for treating patients having various types and sizes of plaque at the CCA bifurcation and particularly those causing severe stenosis. For example, in the case of severe stenosis, one common procedure is carotid endarterectomy in which the plaque is removed surgically after opening the vessel. Another procedure is carotid stenting (also referred to as scaffolding) that involves placement of a metal stent (or scaffold) within the stenosed artery to open the vessel and provide a means of holding the plaque against the arterial wall. One particular advantage of using metal stents is that metal stents are radio-opaque which facilitates deployment procedures as they are visible with imaging equipment.

Importantly, in cases where carotid stenting is performed using a metal stent, the physician must consider the short-term and long-term risks and benefits of deploying a metal stent to treat the particular plaque/thrombus characteristics. One important consideration is that once a metal stent has been deployed, it cannot be removed; hence future treatment options are thereafter reduced when a metal stent has been used. Permanent placement of a metal carotid stent can provide positive benefits of opening a vessel and thus improving blood flow whilst reducing the risk of the plaque breaking free but it can also result in long-term complications such as in-stent stenosis. If a longer term complication does arise, there are then fewer options available.

In general, when a patient has exhibited symptoms, the degree of stenosis of the vessel due to plaque plays a major role in decision making regarding intervention (surgery or stenting). In addition, presence of symptoms related to the plaque/thrombus are important as well. This approach is backed by several randomized controlled trials. For example, for symptomatic patients with >70% stenosis, carotid endarterectomy has shown clear benefit.

There is also increasing data for intervention in symptomatic patients with 50-69% stenosis as well.

For asymptomatic patients with severe stenosis, there is quite a bit of variation of practice around the world as the data is equivocal. In such situations, other factors may come into play such as patency of the circle of Willis, patients' wishes, surgeon/interventionist perceived procedural risk amongst other factors.

As noted above, when a patient has exhibited symptoms, and upon diagnosis, the plaque/thrombus shows relatively low stenosis (<50%), the plaque may also have an unstable appearance where a physician may consider that the risk of the plaque/thrombus breaking free within a relatively short time frame is reasonably high.

There are a number of techniques that help diagnose unstable plaque. It has been shown that plaques may get inflamed and become unstable (such plaques may show enhancement of high-resolution contrast enhanced MR imaging). Hemorrhage into the plaque may also lead to unstable plaque. However, in a significant number of cases, an unstable plaque may “settle down” wherein, over a period of time, the risk of it breaking free becomes lower. It is in certain presentations of those unstable plaques that the present invention is directed.

U.S. provisional application 62/846,467 describes the use of resorbable stents to treat unstable plaque. Generally, while resorbable stents can be engineered to have an outward spring pressure sufficient to properly deploy the stent, in some cases it may be desired or necessary to be able to apply additional outward force to deploy and position the stent.

Accordingly, there has been a need for improved treatment options for unstable plaques that in particular may provide a temporary solution to stabilize the plaque whilst maintaining the potential for a surgeon to conduct future treatments.

### 3. Aneurysms

As described in U.S. patent application Ser. No. 16/239,296, an aneurysm is a blood-filled balloon-like bulge in the wall of a blood vessel, typically caused by flowing blood forcing a weakened section of the blood vessel wall outwards. Aneurysms can occur in any blood vessel but can be particularly problematic when they occur in a cerebral artery. Known as an intracranial or cerebral or brain aneurysm, if a brain aneurysm ruptures, it can lead to a hemorrhagic stroke and potentially cause death or severe disability. The risk of rupture increases with the size of the aneurysm. Most people with un-ruptured brain aneurysms do not have any symptoms and the aneurysm goes undetected. If the aneurysm is by chance detected, which often occurs incidentally, it may be desirable to treat the aneurysm to prevent it from growing, thereby reducing the risk of rupture.

When a patient presents to the hospital with a ruptured brain aneurysm: known as sub-arachnoid hemorrhage (SAH), it is a serious medical emergency. Ruptured aneurysms have a high likelihood of re-rupture which can have devastating consequences. As such, ruptured aneurysms need to be treated as a surgical emergency.

Brain aneurysms 10 develop in various shapes and sizes as shown in FIGS. 3A, 3B, 3C and 3AA with each aneurysm generally characterized by a neck 12 that opens from an artery 14 into an enlarged capsular structure or body. An aneurysm generally has a neck diameter ND, internal radius R and neck angle NA. FIGS. 3A (side view) and 3AA (end view) show the most common type namely a saccular aneurysm that is a “berry-like” bulge or sac that occurs in an artery. In this example, the neck diameter is relatively small compared to the internal radius and the neck angle is less than 90 degrees. FIG. 3B shows a different aneurysm structure having a less spherical shape and that is characterized by a wider neck and a neck angle around 90 degrees. FIG. 3C shows an aneurysm structure where the neck diameter is also greater relative to the internal radius and the neck angle is greater than 90 degrees on at least one side of the aneurysm. Variations in these general types include eccentrically inclined aneurysms (not shown). As will be discussed in greater detail below, the treatment of each of these aneurysms is different.

Generally, the size of the neck typically varies from 2-7 mm and the internal diameter (2 times internal radius) may vary from 3-8 mm. Some aneurysms may also have an irregular protrusion of the wall of the aneurysm, i.e. a “daughter sac”.

The size, shape and location of a brain aneurysm influence the availability and type of treatment. Historically, some brain aneurysms were treated surgically by clipping or closing the base or neck of the aneurysm. Due to the risks and invasiveness of open brain surgery, treatment has moved towards less invasive intravascular techniques. With intravascular techniques, a microcatheter is inserted into the arterial system of a patient, usually through the groin, and threaded through the arterial system to the site of the aneurysm. With one technique, as shown in FIG. 4A, a wire 15 is pushed from a microcatheter 16 and coiled into the body of the aneurysm, so as to pack the aneurysm body with a coil of wire. This wire coil 15 is subsequently detached from the microcatheter by known techniques to enable the microcatheter and remaining wire within the microcatheter to be withdrawn. The wire coil prevents or slows the flow of blood into the aneurysm, causing a thrombus to form in the aneurysm and which then ideally prevents the aneurysm from growing and/or rupturing. During placement and subsequently, it is important that the coil stays within the aneurysm body and does not protrude into the artery. Therefore, this endovascular coiling technique, works best in aneurysms that have narrow necks as shown in FIG. 3A and more specifically with neck diameters less than approximately <4 mm, so as to keep the coiled wire within the aneurysm body

In aneurysms with slightly wider necks, that is similar to an aneurysm as shown in FIG. 3B, balloon-assisted coiling may be used to prevent the coil from protruding into the artery. As shown in FIGS. 4B-4E, a first catheter 16 containing a wire 15 is inserted into the aneurysm body 10. A second catheter 18 having a balloon 20 is placed in the artery adjacent the neck 12 of the aneurysm. As the wire 15 is coiled into the aneurysm, the balloon 20 is temporarily inflated to keep the coiled wire 15 within the aneurysm body. After coiling is complete, or after enough wire has been coiled to keep the wire in place, the balloon is deflated and removed from the artery. One of the risks associated with this type of procedure is that the microcatheter may be too rigid because of the pressure from the balloon and hence may cause the aneurysm to rupture. Other risks are the presence of an inflated balloon in the parent vessel that can lead to thrombus formation. Rarely the vessel may rupture because of over-inflation of the balloon. Most importantly, there is a chance that the coils may prolapse out of the aneurysm once the balloon has been deflated.

In another approach called stent assisted coiling, a stent is placed into the parent vessel preventing the prolapse of the coils. It has some of the disadvantages of balloon assisted coiling but in addition, the other problem is that stents are quite thrombogenic and hence, patients need to be placed on blood-thinners in preparation for stent placement. Of note, some patients have resistance to different blood thinners further adding to the complexity. In addition, generally speaking it is difficult to use stent assisted coiling in acutely ruptured aneurysms as there isn't sufficient time for the blood thinners to act and in addition blood thinners may not be safe in the presence of SAH.

In another endovascular treatment option, instead of a coiled wire, a pre-formed and compressed/collapsed wire mesh ball 22 is pushed out of the catheter and deployed into the body of the aneurysm 10 as shown in FIG. 5A. In this case, the physician chooses a mesh ball size that will best fit within the aneurysm when expanded. Generally, preformed and compressed wire mesh balls are spherical and have specific diameters that can fit within an aneurysm. When deployed and detached, like the coiled wire, the mesh ball seals and/or prevents or slows the flow of blood into the aneurysm, causing a thrombus to form in the aneurysm. This approach typically works best in aneurysms that are more spherical in shape and have a narrow neck to keep the mesh ball within the aneurysm body. However, as shown in FIG. 5B, if the neck is wide and the mesh ball is substantially spherical, regions of the aneurysm may not be completely filled which can result in unfilled pockets 10a, 10b such that if turbulent blood flow is created in those regions, it can result in growth of the aneurysm. In addition, there is also a possibility of aneurysm rupture and thrombus formation that can subsequently break away and cause stroke.

In another intravascular treatment approach for aneurysms as shown in FIG. 6A, a tubular stent 24, i.e. a metal mesh device in the shape of a tube, is placed inside the artery at the site of the aneurysm to cover the neck of the aneurysm. The stent diverts the flow of blood away from the aneurysm, allowing a thrombus to form in the aneurysm. Hence, these devices are often referred to as “flow diverters”. Often the aneurysm will shrink over time after the stent is in place. A stent 24 is particularly useful for large aneurysms and/or aneurysms with wide necks and/or irregular shaped bodies. A stent may be used on its own or in conjunction with another device like a coiled wire or mesh ball. The stent can help keep the coiled wire or mesh ball within the aneurysm body if the aneurysm has a wide neck. The disadvantages of a stent are that it creates a large area of metal within the artery which increases the chance of thrombi forming on the stent. Patients with stents typically need to take antiplatelet medication indefinitely to prevent blood clots from forming and growing. While stents can work well for certain types of aneurysms, particularly ones that are located in straight arterial passageways, they are not ideal for all aneurysms. That is, if there are one or more bifurcations 14a in the arterial vessel near the aneurysm, the stent would block off flow to the other vessel and would therefore not be suitable for use if the aneurysm is located near a bifurcation 14a as shown in FIG. 6B.

In addition, once one of these flow diverters are placed across the neck of the aneurysm, it practically obviates any future option for an alternative treatment into the sac of the aneurysm as the pores of the flow diverter are so small that no device can be introduced through it.

Accordingly, there continues to be a need for improved systems and methods for treating brain aneurysms, particularly ones that are irregular shaped and/or have wide necks. There is also a need for treating brain aneurysms that are at arterial sites with bifurcations nearby.

Furthermore, there continues to be a need for systems and methods for the treatment of aneurysms where resorbable stents are utilized.

### SUMMARY OF THE INVENTION

According to a first aspect of the invention, a method of deploying a resorbable stent (RS) in an arterial vessel of a patient is provided, comprising the steps of: advancing a catheter system operatively retaining a collapsed RS to a desired location within the patient; and deploying and releasing the RS within the vessel; where the RS has: a collapsible cylindrical body for compressed containment within the catheter system; sufficient self-expansion properties enabling the RS to engage with the arterial vessel upon deployment; and resorption properties where the RS is resorbed over a resorb time.

In one embodiment, the method is for treatment of an unstable plaque/web/thrombus in a patient with or without significant stenosis, the method to stabilize the unstable plaque/web/thrombus for a therapeutically effective time period and the desired location is at or adjacent to a bifurcation of a Common Carotid Artery (CCA) into an Internal Carotid Artery (ICA) and the step of deploying further includes: deploying the RS over the unstable plaque/web/thrombus; and where the RS has: a pore size sufficiently small to prevent embolization of plaque/thrombus fragments after deployment.

In another embodiment, the method is for treatment of an arterial aneurysm and the step of deployment includes deploying the RS over an aneurysm neck and where the RS has a pore size sufficiently small to prevent blood flow into the aneurysm after deployment.

Various embodiments of the methods further may comprise various steps including:
substantially arresting blood flow adjacent to the desired location prior to deploying the RS;
advancing a balloon guide catheter (BGC) proximal to the desired location and inflating a first balloon to occlude blood flow through the desired location;
advancing a micro-balloon (MB) through the BGC and inflating the MB in an external carotid artery (ECA) adjacent a CCA bifurcation; and/or,
establishing retrograde flow through the BGC to remove debris adjacent the CCA bifurcation.

The RS may have a pore size enabling the RS to act as a distal protection device (DPD) during RS deployment.

The resorb time may be variable and designed to be one week or less; one month or less; two months or less or longer.

The RS may be a drug-eluting RS that may be adapted to release one or more anti-mitotic drugs and/or one or more anti-thrombogenic drugs and/or one or more anti-inflammatory drugs such as heparin.

The RS may be adapted for reduced thrombogenicity.

The RS may have a taper to accommodate for the reduction of diameter between the CCA and ICA.

In another aspect, the invention provides a method of deploying a resorbable stent (RS) in an arterial vessel of a patient, comprising the steps of: advancing a catheter system operatively retaining a collapsed co-axial stent system (COSS) having an outer resorbable stent (RS) and a metal stent (MS); deploying the co-axial stent system (COSS) from the catheter at a desired location within the patient and releasing the RS; allowing sufficient time for the MS to assist in seating the RS in the vessel; and, re-sheathing the MS into the catheter where the RS has: a collapsible cylindrical body for compressed containment within the catheter system; and, resorption properties where the RS is resorbed over a resorb time and the MS has: a collapsible cylindrical body for compressed containment within the catheter system and inside the RS; and sufficient self-expansion properties enabling the MS to bias the RS against the arterial vessel upon deployment.

The method may be used for treatment of an unstable plaque/web/thrombus in a patient with or without significant stenosis, the method to stabilize the unstable plaque/web/thrombus for a therapeutically effective time period and the desired location is at or adjacent to a bifurcation of a Common Carotid Artery (CCA) into an Internal Carotid Artery (ICA) and where the step of deploying further includes: deploying the RS over the unstable plaque/web/thrombus; and where the RS has: a pore size sufficiently small to prevent embolization of plaque/thrombus fragments after deployment.

The method may be used for treatment of an arterial aneurysm and the step of deployment may include deploying the RS over an aneurysm neck and where the RS has a pore size sufficiently small to prevent blood flow into the aneurysm after deployment.

The method may include various steps including:
substantially arresting blood flow adjacent to the desired location prior to deploying the COSS;
advancing a balloon guide catheter (BGC) proximal to the desired and inflating a first balloon to occlude blood flow;
advancing a micro-balloon (MB) through the BGC and inflating the MB in an ECA adjacent a CCA bifurcation and/or,
establishing retrograde flow through the BGC to remove debris adjacent the CCA bifurcation.

In another aspect the invention describes the use of a resorbable stent to stabilize an unstable plaque for a therapeutically effective time period in a patient at or adjacent to a bifurcation of a Common Carotid Artery (CCA) into an Internal Carotid Artery (ICA) and an External Carotid Artery (ECA) (the CCA bifurcation) in a patient.

In another aspect the invention describes the use of a resorbable stent to stabilize an aneurysm for a therapeutically effective time period in a patient.

In another aspect, the invention describes the use of a co-axial stent system (COSS) at a desired location in an arterial vessel, the COSS having a combined inner metal stent (MS) and outer resorbable stent (RS) to a) stabilize an unstable plaque for a therapeutically effective time period in a patient at or adjacent to a bifurcation of a Common Carotid Artery (CCA) into an Internal Carotid Artery (ICA) and an External Carotid Artery (ECA) (the CCA bifurcation) or b) to occlude an aneurysm neck in a patient.

In one embodiment, the MS is re-sheathed and removed after deployment of the RS.

In one embodiment, the MS is detached after deployment of the RS and remains at the desired location.

In another aspect the invention provides a kit for the treatment of an unstable plaque or an aneurysm at a desired location in a patient, the kit comprising: at least one guide catheter (GC) for placement proximal to the desired location; at least one guide wire for placement distal to the desired location; at least one microcatheter for placement distal to the desired location over the guide wire; at least one resorbable stent (RS) assembly for placement adjacent to the desired location and deployable through the at least one microcatheter each RS assembly having a RS to stabilize the unstable plaque or aneurysm for a therapeutically effective time period and resorbable into the patient over a resorb time. The GC may be at least one balloon guide catheter (BGC) for occluding blood flow and may include at least one micro-balloon (MB) for occluding blood flow through the ECA.

A kit may include at least two resorbable stent assemblies each having a resorbable stent, and where the resorbable stents have at least one structural and/or functional property different from each other, selected from any one of or a combination of stent diameter, stent length, stent taper, stent compressive stiffness, stent pore size; stent drug coating and stent resorb time.

In another aspect, the invention provides a resorbable stent (RS) for deployment within an arterial vessel of a patient at a desired location, the RS comprising: a cylindrical body having a plurality of pore openings in the range of 110-250 microns diameter and a void space of greater than 50% of the cylindrical body, the cylindrical body collapsible within a microcatheter and deployable from the microcatheter for placement with the arterial vessel at the desired location and wherein the cylindrical body is self-expanding upon deployment within an artery and resorbable into the patient after deployment.

The resorbable stent may include a cylindrical body comprising a weave of poly lactic-co-glycolic acid fibers, the fibers having a diameter in the range of 30-50 microns.

The resorbable stent may have a rate of resorption proportional to blood flow through stent tines wherein regions of the stent subjected to higher blood flow will resorb faster than regions of the stent having lower blood flow.

The resorbable stent may have resorb properties where the cylindrical body resorbs progressively along exposed edges of the cylindrical body not in contact with a vessel wall towards a vessel wall so as to maintain a structural integrity of the cylindrical body during resorption.

The resorbable stent may have resorb properties such that during resorption of exposed edges of the cylindrical body not in contact with a vessel wall, surfaces of the cylindrical body in contact with a vessel wall endothelialize and do not resorb.

In another aspect, the invention provides a co-axial stent system (COSS) comprising: a catheter system for retaining: a collapsible resorbable stent (RS) having: a collapsible cylindrical body for compressed containment within the catheter system; and, resorption properties where the RS is resorbable within a patient over a resorb time; a collapsible metal stent (MS) affixed to a stent wire (SW) passing through catheter system, the MS having: a collapsible cylindrical body for compressed containment within the catheter system and the RS; sufficient self-expansion properties enabling the MS to bias the RS against the arterial vessel upon deployment; wherein the MS may be unsheathed and re-sheathed from the catheter system and wherein upon deployment of the RS and re-sheathing of the MS, the RS remains deployed within an arterial vessel.

In another aspect, the invention provides a co-axial stent system (COSS) comprising: a catheter system for retaining: a collapsible resorbable stent (RS) having: a collapsible cylindrical body for compressed containment within the catheter system; and, resorption properties where the RS is resorbable within a patient over a resorb time; a collapsible metal stent (MS) affixed to a stent wire (SW) passing through catheter system, the MS having: a collapsible cylindrical body for compressed containment within the catheter system and the RS; sufficient self-expansion properties enabling the MS to bias the RS against the arterial vessel upon deployment; wherein the MS may be unsheathed and re-sheathed from the catheter system and wherein upon deployment of the RS and re-sheathing of the MS, the RS remains deployed within an arterial vessel.

### BRIEF DESCRIPTION OF THE DRAWINGS

Various objects, features and advantages of the invention will be apparent from the following description of particular embodiments of the invention, as illustrated in the accompanying drawings. Similar reference numerals indicate similar components:

FIG. 1 is a schematic diagram of a CCA bifurcation.

FIG. 2 is a schematic diagram of the anatomy of a typical Circle of Willis.

FIGS. 3A, 3B, 3C and 3AA are schematic diagrams of different aneurysm structures showing typical variations in neck diameter and neck angle.

FIGS. 4A-4E are schematic diagrams of wire coiling methodologies for treating aneurysms including narrow neck and wider neck aneurysms with a balloon catheter (FIGS. 4B-4D) and without a balloon catheter (FIG. 4A) in accordance with the prior art.

FIGS. 5A and 5B are schematic diagrams showing the methodology of placing and deploying a wire mesh ball for the treatment of an aneurysm in accordance with the prior art.

FIGS. 6A and 6B are schematic diagrams showing a methodology of placing a wire mesh stent for the treatment of an aneurysm away from a bifurcation (FIG. 6A) and near a bifurcation (FIG. 6B) in accordance with the prior art.

FIG. 7 is a flow chart of a method for treatment of an unstable plaque, according to one embodiment of the invention.

FIG. 8 is a schematic diagram of a CCA bifurcation showing an unstable plaque and a balloon guided catheter (BGC) inserted in a CCA with a first balloon being inflated, according to one embodiment.

FIG. 9 is a schematic diagram of the CCA bifurcation of FIG. 8, with the BGC extending into the ECA, the first balloon being fully inflated, and a second balloon being inflated.

FIG. 10 is a schematic diagram of the CCA bifurcation of FIG. 9, with the second balloon fully inflated and a guide wire inserted through an aperture of the BGC and into the ICA, past the unstable plaque.

FIG. 10A is a schematic diagram showing a combined balloon guide catheter (BGC) and micro-balloon (MB).

FIG. 11 is a schematic diagram of the CCA bifurcation of FIG. 10, showing a microcatheter extending along the guide wire.

FIG. 12 is a schematic diagram of the CCA bifurcation of FIG. 11, with the guide wire removed.

FIG. 13 is a schematic diagram of the CCA bifurcation of FIG. 12, showing a stent assembly that has been advanced inside the microcatheter.

FIG. 14 is a detailed view of a portion of a proximal end of a stent assembly as shown in FIG. 13.

FIG. 15 is a schematic diagram of the CCA bifurcation of FIG. 13, showing a resorbable stent of the stent assembly being deployed and acting as a distal protection device.

FIG. 15A is a schematic diagram showing a resorbable stent being deployed over an unstable plaque.

FIG. 16 is a schematic diagram of the CCA bifurcation of FIG. 15, showing the resorbable stent being further deployed.

FIG. 17 is a schematic diagram of the CCA bifurcation of FIG. 16, showing the resorbable stent in the deployed position with the BGC and the microcatheter having been removed.

FIG. 18 is a schematic diagram of a resorbable stent being deployed without flow cessation.

FIGS. 19A-19H are schematic diagrams of a co-axial stent system (COSS) and a method of deployment in accordance with one embodiment of the invention.

FIGS. 20A and 20B are schematic diagrams of a co-axial stent system (COSS) and a method of deployment in accordance with one embodiment of the invention.

FIGS. 21A, 21A1, 21B, 21C and 21C1 are schematic cross-sectional diagrams of a resorbable stent showing placement and the progression of resorption in accordance with one embodiment of the invention.

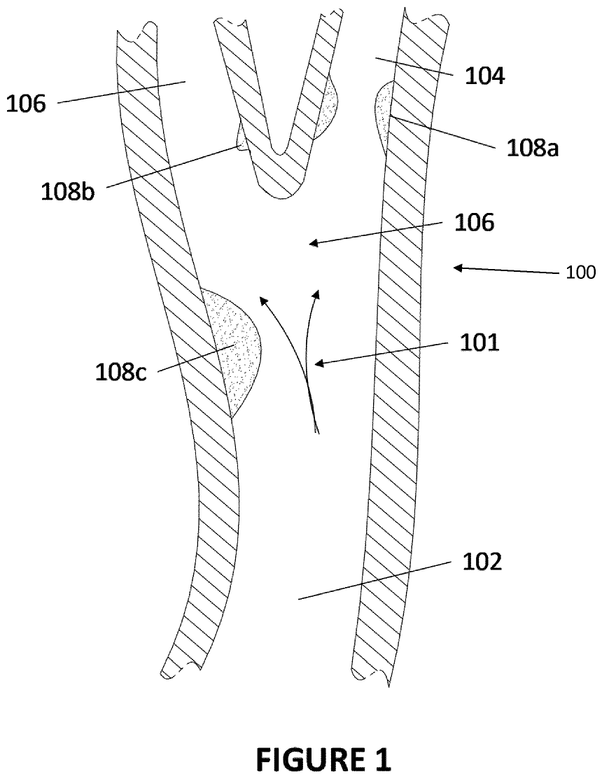

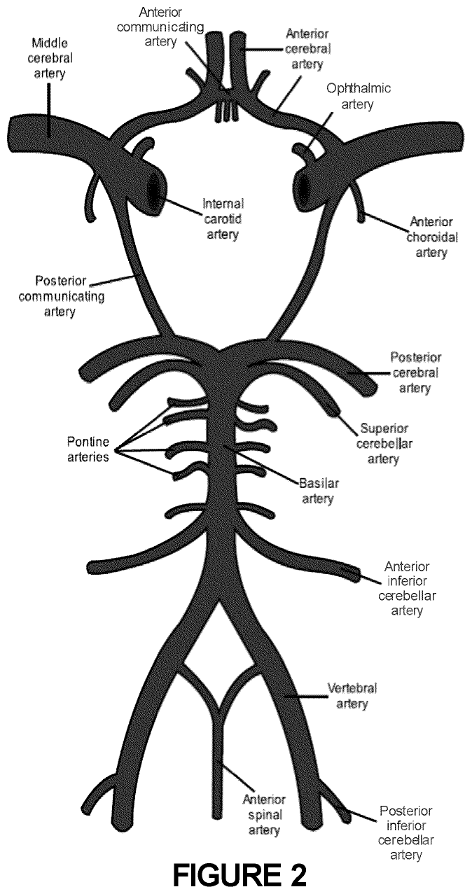

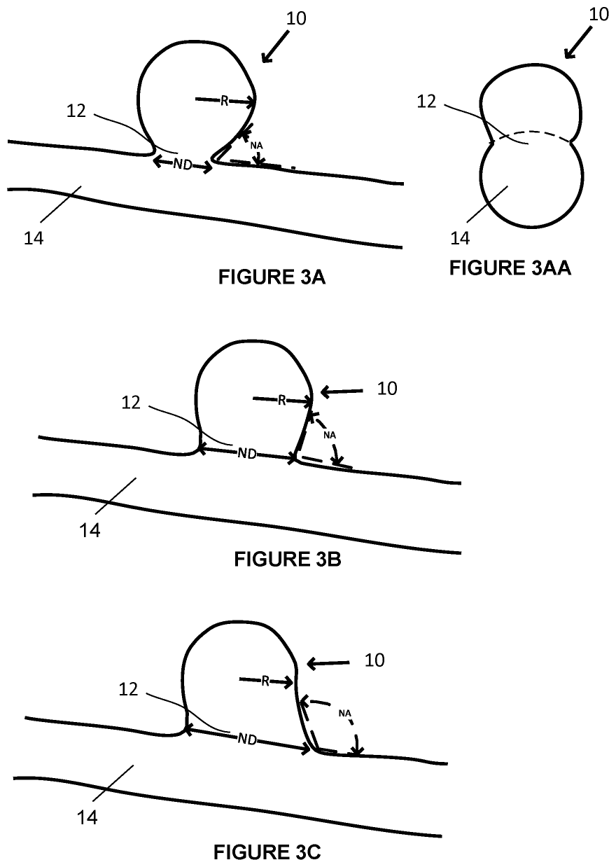

### DETAILED DESCRIPTION OF THE INVENTION

### 4. Introduction and Rationale

The inventor understood that patients may be at high risk for AS/TIAs if they have aggressive-looking or unstable plaque at the CCA bifurcation even if they don't have significant carotid stenosis.

An unstable plaque will typically have produced symptoms in the ipsilateral circulation (e.g. amaurosis fugax, TIA) and have an irregular shape and generally be adhered to a smaller proportion of the arterial vessel as compared to an atherosclerotic plaque where the degree of stenosis is greater than 50%. Due to its irregular shape, blood flow around the unstable plaque may be turbulent which may lead to the plaque, or portions of the plaque, breaking free.

The diagnosis of unstable plaque may be made using a combination of factors after a patient has exhibited various symptoms. These factors include: presence of irregular plaque at the ipsilateral carotid origin determined by imaging; absence of any other risk factors (e.g. cardiac issues such as atrial fibrillation); strokes limited to that circulation on diffusion MRI; presence of blood products within the plaque or enhancement of the plaque on high resolution MRI; and presence of ‘donut sign’ on CT angiography.

Modification in the shape or morphology of the plaque over short term repeat imaging is another pointer.

Current literature does not advocate procedures to acutely manage these plaques to immediately reduce the risk of sudden embolic stroke without potentially introducing long term risks. Further, it is not uncommon for an unstable plaque to stabilize or settle down by itself over the next several weeks. Therefore, patients with unstable plaques may be managed with heparin and other anti-coagulation drugs in hopes that the unstable plaque with stabilize before it embolizes into the distal circulation.

The present inventor, having a background in the medical treatment of strokes and TIAs, is familiar with technological developments occurring in this field in recent years. The inventor recognized that further options must be developed for the acute treatment of AS/TIAs that do not introduce long term health risks. The inventor realized that it is desirable to stabilize an unstable plaque in the short term to minimize the risk of it suddenly breaking free without introducing further or long-term risks.

The present inventor has also recognized that the placement of resorbable stents may require additional outward force/pressure to ensure proper deployment.

Further still, the present inventor has recognized that improved placement of resorbable stents is also applicable to the placement of flow diverters in the treatment of aneurysms including wide-neck aneurysms.

### 5. Terminology

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items.

Spatially relative terms, such as “distal”, “proximal”, “forward”, “rearward”, “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a feature in the figures is inverted, elements described as “under” or “beneath” other elements or features would then be oriented “over” the other elements or features. Thus, the exemplary term “under” can encompass both an orientation of over and under. A feature may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

It will be understood that when an element is referred to as being “on”, “attached” to, “connected” to, “coupled” with, “contacting”, etc., another element, it can be directly on, attached to, connected to, coupled with or contacting the other element or intervening elements may also be present. In contrast, when an element is referred to as being, for example, “directly on”, “directly attached” to, “directly connected” to, “directly coupled” with or “directly contacting” another element, there are no intervening elements present.

It will be understood that, although the terms “first”, “second”, etc may be used herein to describe various elements, components, etc., these elements, components, etc. should not be limited by these terms. These terms are only used to distinguish one element, component, etc. from another element, component. Thus, a “first” element, or component discussed herein could also be termed a “second” element or component without departing from the teachings of the present invention. In addition, the sequence of operations (or steps) is not limited to the order presented in the claims or figures unless specifically indicated otherwise.

Other than described herein, or unless otherwise expressly specified, all of the numerical ranges, amounts, values and percentages, such as those for amounts of materials, elemental contents, times and temperatures, ratios of amounts, and others, in the following portion of the specification and attached claims may be read as if prefaced by the word “about” even though the term “about” may not expressly appear with the value, amount, or range. Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques.

Unless otherwise defined, all technical and scientific terms used herein have the same meaning as commonly understood by one of ordinary skill in the art to which this invention belongs.

Various aspects of the invention will now be described with reference to the figures. The invention may, however, be embodied in many different forms and should not be construed as limited to the embodiments set forth herein; rather, these embodiments are provided so that this disclosure will be thorough and complete, and will fully convey the scope of the invention to those skilled in the art.

### 6. Systems and Methods for Treatment of Unstable Plaque

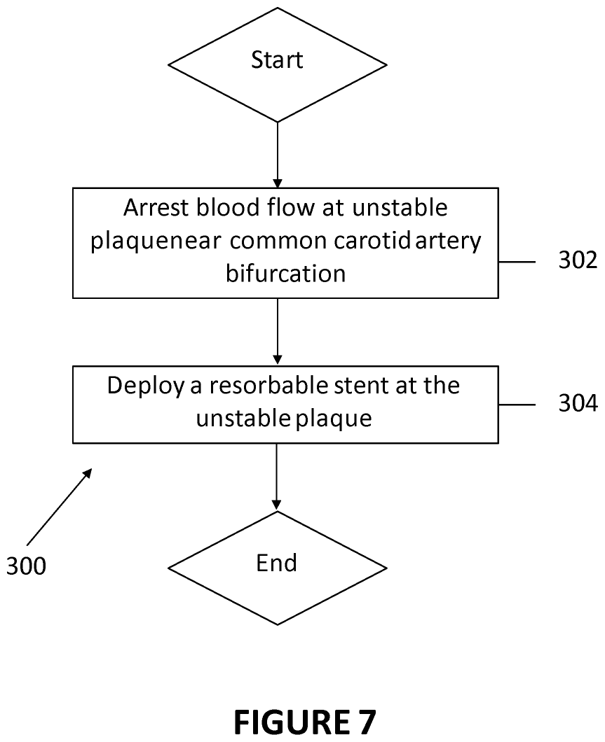

An example method for the treatment of an unstable plaque at the CCA bifurcation will now be described with reference to FIGS. 7 to 18. In this description, a resorbable stent is resorbable and has the following properties:
resorbable over a period of time (for example 1 week to a few months);
self-expanding upon deployment from a catheter;
outward spring strength to push against an arterial wall independently and/or in conjunction with a co-axial stent system as described herein,
low porosity relative to the size of potential emboli breaking off the surface of the unstable plaque whilst enabling blood cells to pass through the stent;
a typical pore size may be 110-250 microns);
and optionally may be:
tapering to enable effective placement in tapered arterial vessels;
having resorption characteristics that are related to the flow rate of blood over or through the resorbable stent; and/or
a substrate for local drug delivery or to reduce thrombogenicity.

FIG. 7 shows a flow chart of a method 300 for treatment of an unstable plaque, according to one embodiment. The method includes, at step 302, substantially arresting blood flow adjacent to the CCA bifurcation and the unstable plaque and at step 304, deploying a resorbable stent over the unstable plaque to stabilize the unstable plaque for a therapeutically effective time period and wherein the stent is resorbed over a resorb time.

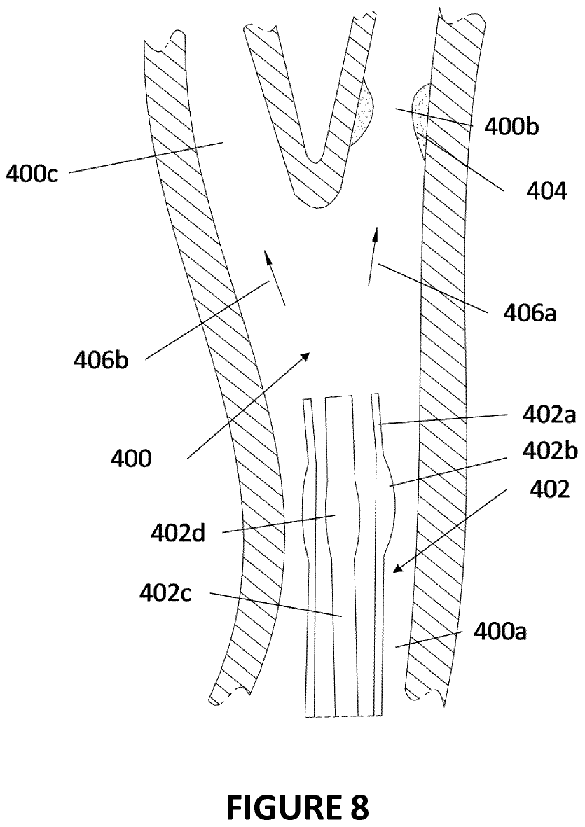

The method 300 will be further illustrated with regard to the example steps shown in FIGS. 7 to 18. FIGS. 8 to 18 show similar features. Features that are common between FIGS. 8 to 18 have not necessarily been relabeled for clarity of the drawings.

FIG. 8 is a schematic of a CCA bifurcation 400 having a CCA 400a, an ICA 400b and an ECA 400c. FIG. 8 also shows an unstable plaque 404 located in the ICA 400b, and a balloon guide catheter (BGC) 402 inserted into the CCA 400a and proximal to the CCA bifurcation.

Flow lines 406a, 406b show the direction of blood flow from the CCA 400a to both the ICA 400b and the ECA 400c.

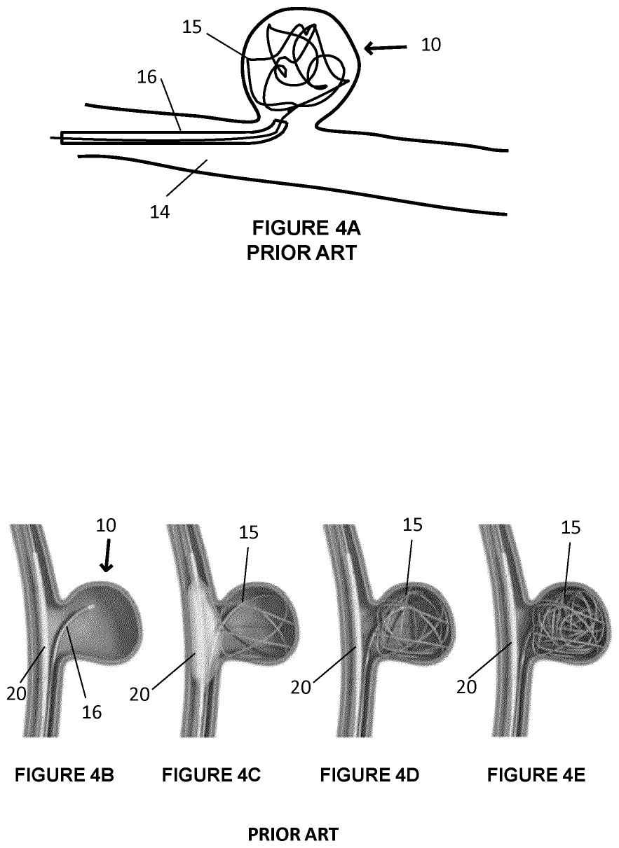

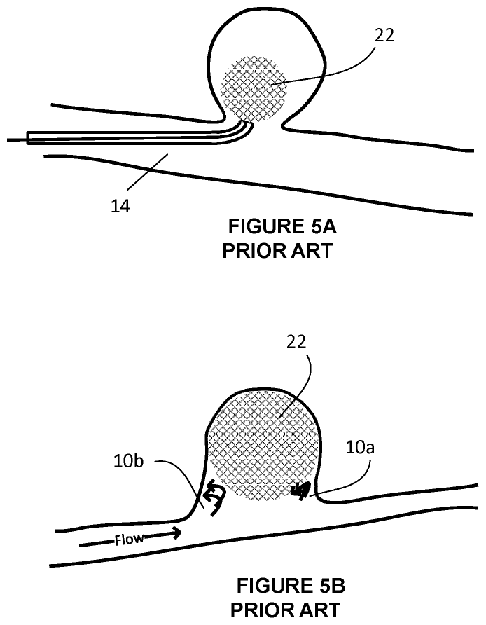

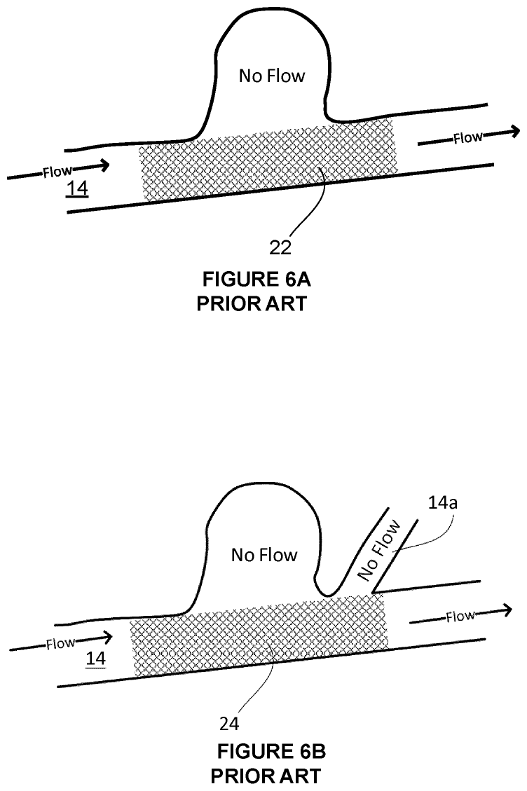

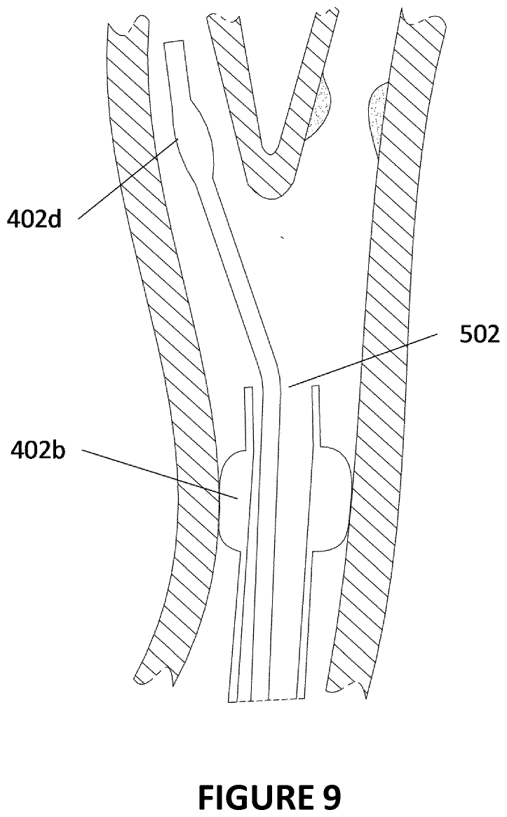

The balloon guide catheter (BGC) 402 includes a first catheter 402a having a balloon 402b. In FIG. 4, the balloon 402b is in the process of being inflated and FIG. 9 shows the balloon fully inflated.

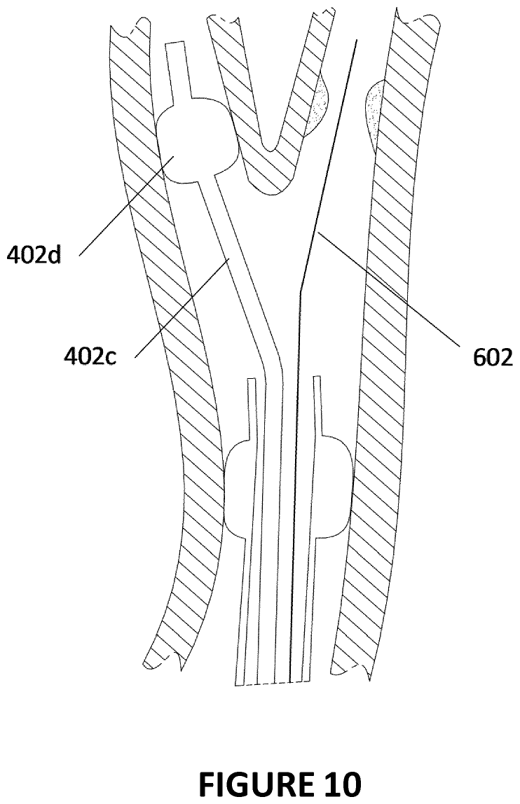

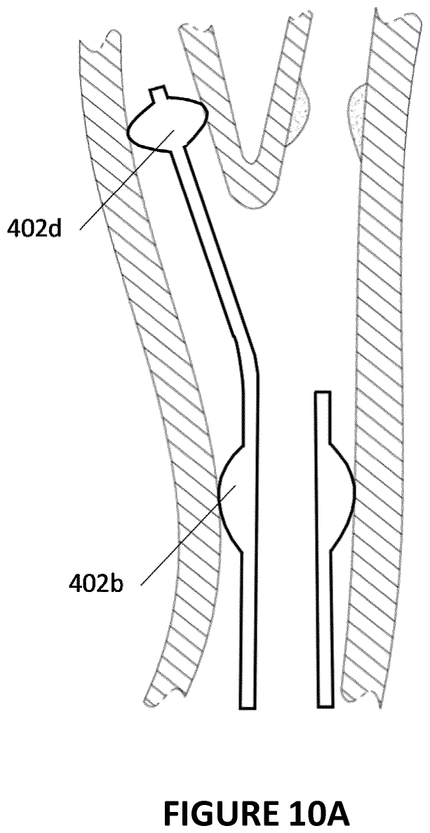

Within the BGC is a micro-balloon (MB) 402d (forming part of a microcatheter 402c such that it can be inserted through the BGC and still leave suitable space for a resorbable stent to be deployed through the BGC). As shown in FIG. 8, the MB is advanced through the first BGC in an uninflated configuration. The MB is expandable to a caliber to completely fill the lumen of the ECA and be occlusive as shown in FIG. 10.

In an alternative design as shown in FIG. 10A, the BGC and MB are constructed as one piece where the MB is attached to the tip of the BGC and both the balloons share a common connection for inflation from the outside. The distance between the tip of the BGC and the distal micro-balloon would typically be 5-10 cm. The purpose of this alternate design is to have greater space within the lumen of the BGC 402b to accommodate the resorbable stent.

The BGC 402 (and MB if a unitary design) may be inserted into the CCA 400a by known techniques. For example, the BGC 402 may be inserted through the aortic arch according to standard procedures. The BGC 402 is then manipulated to be in the CCA 400a proximal to the unstable plaque 404, and the balloon on the BGC 402b is inflated as described above.

Once inflated, the first balloon 402b arrests antegrade flow through the CCA, ICA and ECA.

Turning now to FIG. 9 and FIG. 10, as shown the MB 402d is in a position to be fully inflated and the second catheter 402c of the MB 402d has been advanced through an aperture 502 of the BGC 402a and into the ECA 400c. The MB 402d is in the process of being inflated in FIG. 9. When inflated, the two balloons (BGC and MB) prevent essentially all antegrade flow from the CCA and retrograde flow down the ECA thus providing a substantially zero flow area at the level of the unstable plaque to conduct a stenting procedure.

While flow in the CCA, ICA and ECA on the ipsilateral side has been stopped, flow through the Circle of Willis (COW) and other vessels will usually provide enough circulation to keep the brain alive for a period of time. Moreover, as is understood, there are variations in patients' anatomies that may affect how a surgeon chooses to conduct a procedure having consideration to the specifics of a case. However, generally it is desirable that all procedures be conducted as quickly as possible to minimize the time where blood flow through the ipsilateral CCA is being occluded.

Importantly, the aperture 502 of the BGC allows selective communication between the BGC and the treatment area.

### 7. Stenting Procedures

The stenting procedure is conducted with reference to FIGS. 10-18. FIG. 10 is a schematic diagram of the CCA bifurcation 400, with the MB 402d fully inflated. As noted above, with both the balloons 402b, 402d fully inflated, blood flow adjacent the unstable plaque has been substantially arrested.

With blood flow arrested, a guide wire or microwire 602 (hereinafter referred to as a “guide wire”, for simplicity) is extended though the BGC 402a, through the aperture 502 and into the ICA 400b, past the unstable plaque 404. The guide wire 602 is placed to enable the deployment of a resorbable stent over the plaque as described below.

In various embodiments, the guidewire may have a distal protection device (DPD), such as a basket with small pores that allow blood to go through but would capture any emboli dislodged during the procedure (not shown) to provide an additional level of protection against procedural strokes. However, as explained below the need for DPD is reduced by the stents described herein.

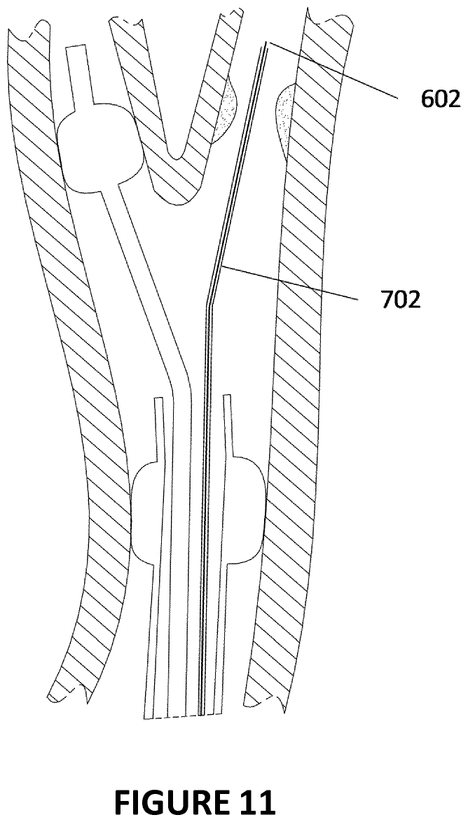

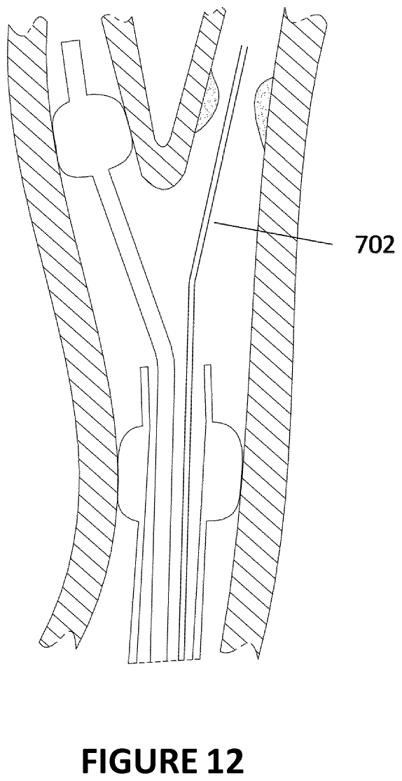

With the guide wire in place, FIG. 11 shows a microcatheter 702 extending over the guide wire 602 to a position distal to the unstable plaque 404. The microcatheter 702 may be advanced over the guide wire 602 by known techniques.

With the microcatheter 702 in place, the guide wire 602 is removed as shown in FIG. 8.

After the guide wire 602 has been removed a resorbable stent 902a, which is part of a stent assembly 902, may be advanced within the microcatheter 702 to a location where the resorbable stent will be deployed, namely at the site of the unstable plaque. FIGS. 9 and 10 show the resorbable stent 902a as part of a stent assembly 902 and will therefore be discussed together.

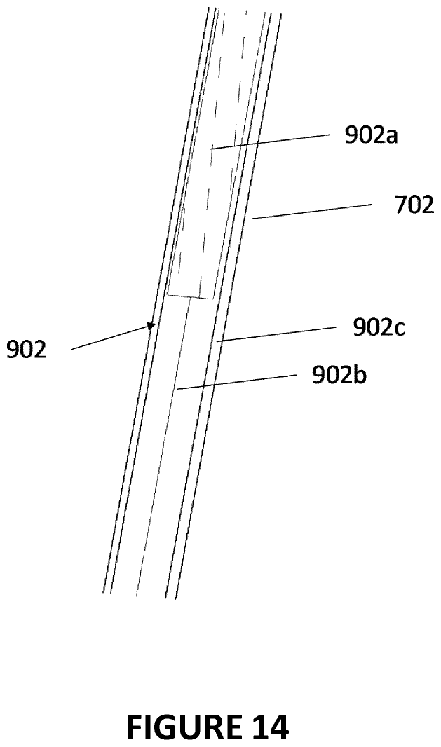

Turning first to FIG. 14, the stent assembly 902 is shown to include a resorbable stent 902a, an engagement or push wire 902b connected to or engageable with the resorbable stent, and a sheath 902c enveloping the resorbable stent. The resorbable stent 902a is in the undeployed position, with the sheath 902c surrounding the resorbable stent. The engagement or push wire 902b is used to hold the resorbable stent 902a in position while the sheath 902c and the microcatheter 702 are removed in the proximal direction.

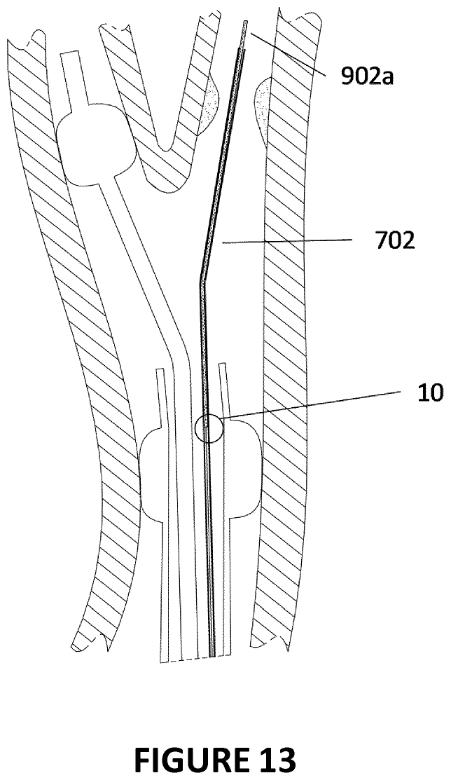

FIG. 13 also shows the stent assembly 902 in a position where the resorbable stent 902a extends slightly beyond the unstable plaque 404.

In the embodiment shown in FIGS. 9 and 10, the resorbable stent 902a is a self-expanding resorbable stent, whereby withdrawing the sheath 902c will deploy the stent by spring energy stored in the compressed stent. Generally, the resorbable stent 902a will be sufficiently flexible to resist substantial deformation when the patent moves their neck.

The resorbable stent 902a may include certain features complementary with its deployment at the unstable plaque 404. For example, the resorbable stent 902a may be made of poly (lactic-co-glycolic) acid (PLGA) or any other material that is sufficiently rigid but may dissolve in the blood stream without deleterious effects. In an embodiment, the resorbable stent 902a may be adapted for reduced thrombogenicity. Certain features of such stents can include stents with specific coatings or geometries. In one embodiment, the resorbable stent 902a has a pore size sufficiently small to prevent small pieces of the plaque emerging through the pores and breaking free whilst providing sufficient outward force to maintain and outward pressure against the plaque and the adjacent arterial walls.

In an embodiment, although not required, the resorbable stent 902a may be a drug-eluting resorbable stent. For example, the drug-eluting resorbable stent may be adapted to release one or more anti-mitotic drugs and/or one or more anti-thrombogenic drugs and/or one or more anti-inflammatory drugs. The anti-inflammatory drugs may include heparin or warfarin, or a combination thereof, which may help stabilize the plaque.

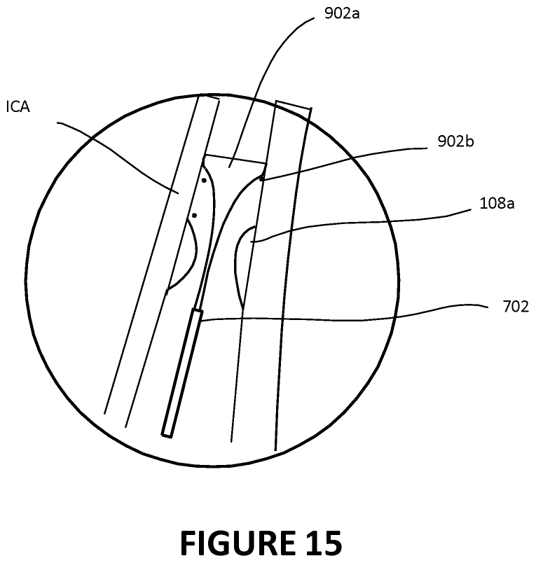

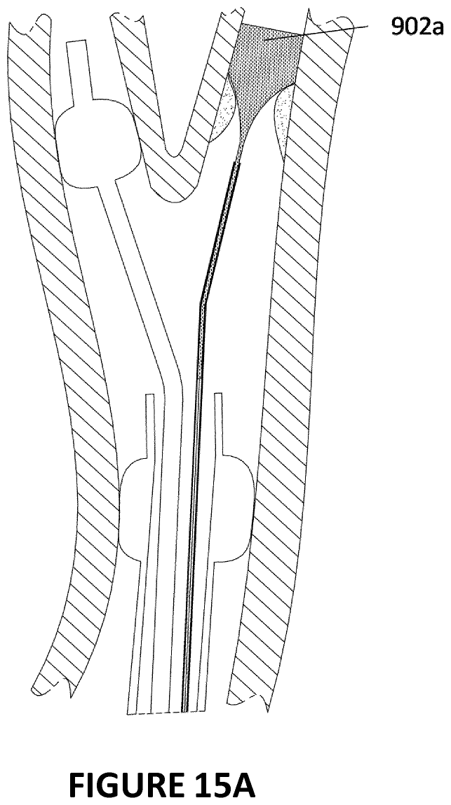

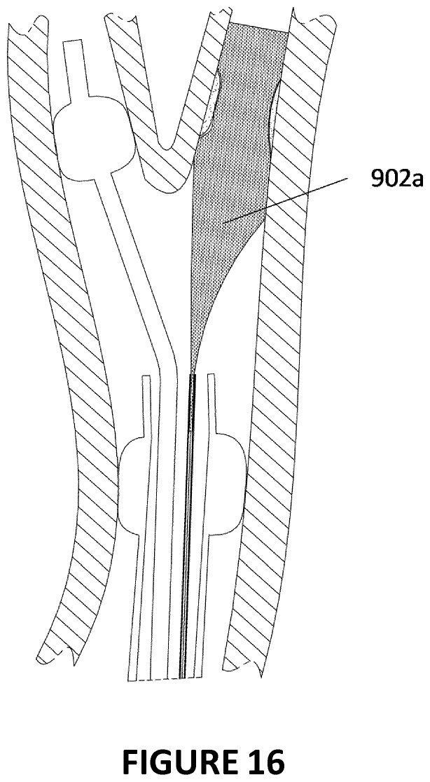

FIGS. 15, 15A and 16 shows the resorbable stent 902a being deployed. Specifically, the sheath 902c and the microcatheter 702 are withdrawn while the resorbable stent 902a is held in position by the engagement or push wire 902b. As the resorbable stent 902a expands it pushes against and/or compresses the unstable plaque 108a, thereby stabilizing the unstable plaque. Once the resorbable stent 902a is fully unsheathed, the engagement/push wire is withdrawn together with the microcatheter.

The resorbable stent 902a may then remain at the site for a therapeutically effective time period and/or until it is resorbed. During the therapeutically effective time period the unstable plaque 404 may convert to atherosclerotic plaque, may dissolve in the blood stream and/or may be absorbed by the blood vessel of the ICA 400b, or a combination thereof. In an embodiment, the therapeutically effective time period and/or resorb time period may be less than one week. In another embodiment, the therapeutically effective time period and/or resorb time period may be less than one month, less than two months or less than three months. The length of the therapeutically effective time period and/or resorb time period may be determined by a number of factors including: how unstable the plaque is; the desired treatment outcome; the type of stent that is deployed; and the postoperative treatment protocol. After the therapeutically effective time period, the resorbable stent 902a may have substantially resorbed into the blood stream.

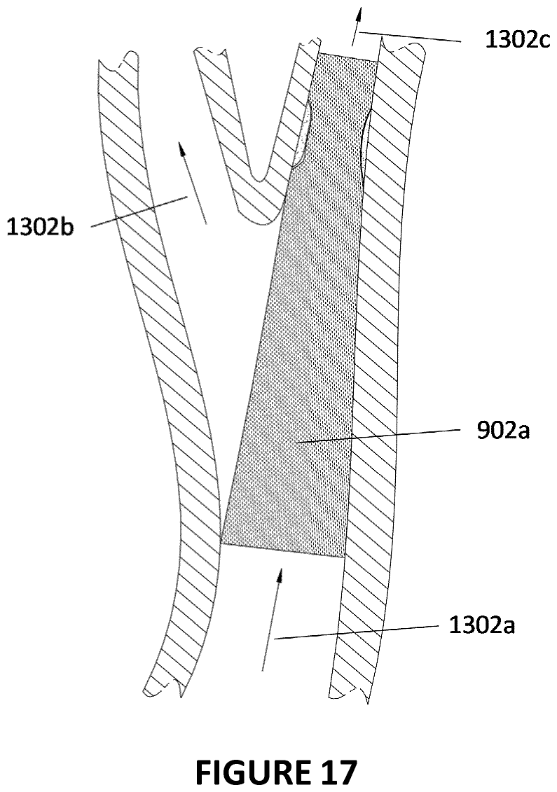

FIGS. 16 and 17 show the resorbable stent 902a deployed or bearing against the unstable plaque. The diameter, circumference and length of the resorbable stent 902a is merely exemplary.

Generally, during and/or after the resorbable stent 902a deployment, debris is removed from the area via suction through the BGC 402. In another embodiment, a filter may be used to remove any accumulated debris.

Once the resorbable stent 902a is deployed, the microcatheter 702 is removed, the first balloon 402b and the MB 402d are deflated and removed, thus re-establishing flow. Blood flow lines 1302a,1302b,1302c show that normal blood flow from the CCA 400a to both the ICA 400b and the ECA 400c has been restored. As shown by the flow lines 1302a,1302c, blood may pass within the deployed resorbable stent 902a.

In the embodiment shown in FIG. 17, the resorbable stent 902a partially occludes the ECA 400c. Specifically, while the resorbable stent extends into the CCA 400a, at least some blood may be able to flow around or over the edges of the resorbable stent 902a and arterial walls and/or through pores in the resorbable stent. In another embodiment, the resorbable stent 902a may completely cover the origin of the ECA 400c, however, blood flow to the ECA is still maintained by virtue of the Circle of Willis and other cross-connections, described above.

Before and during the procedure, an anti-platelet and anti-coagulation drug regime may help reduce the risk that any debris released during the procedure will form a clot.

The procedure (from insertion of the BGC/MB and stent placement to removal), may be accomplished within about 3-5 minutes.

Importantly, the procedure does not affect the ability to do other procedures in the future in the event of stenosis, growth or changes to the plaque at the site and/or a continued unstable appearance of the plaque. That is, to the extent that the stent has dissolved and the plaque has characteristics that may warrant the same or different treatment, these future procedures may be conducted.

### 8. Co-Axial Stent System (COSS)

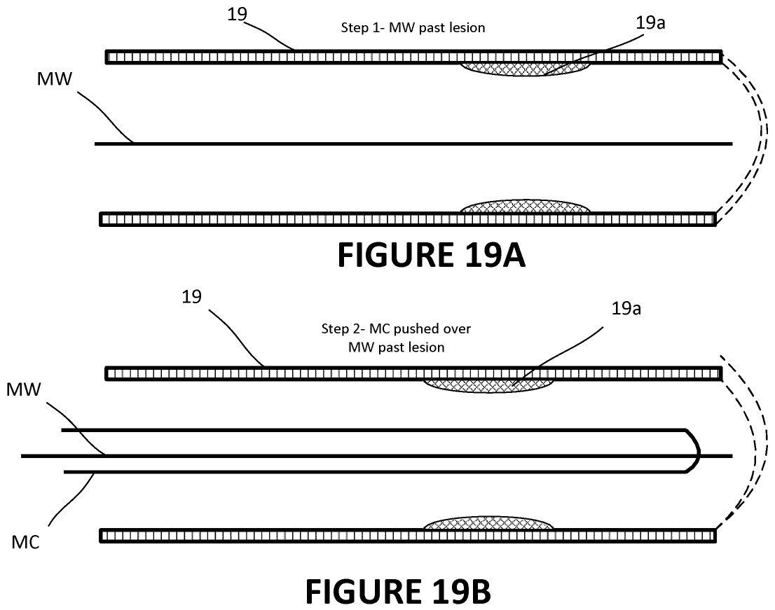

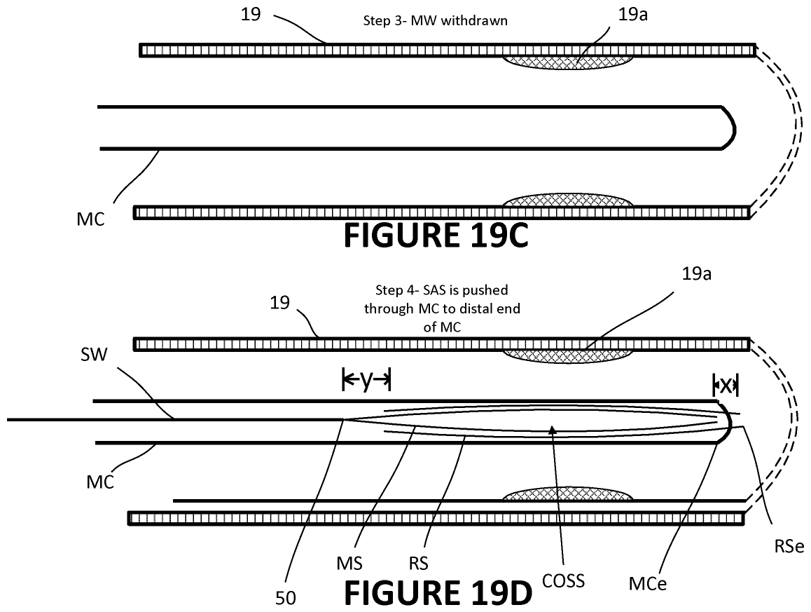

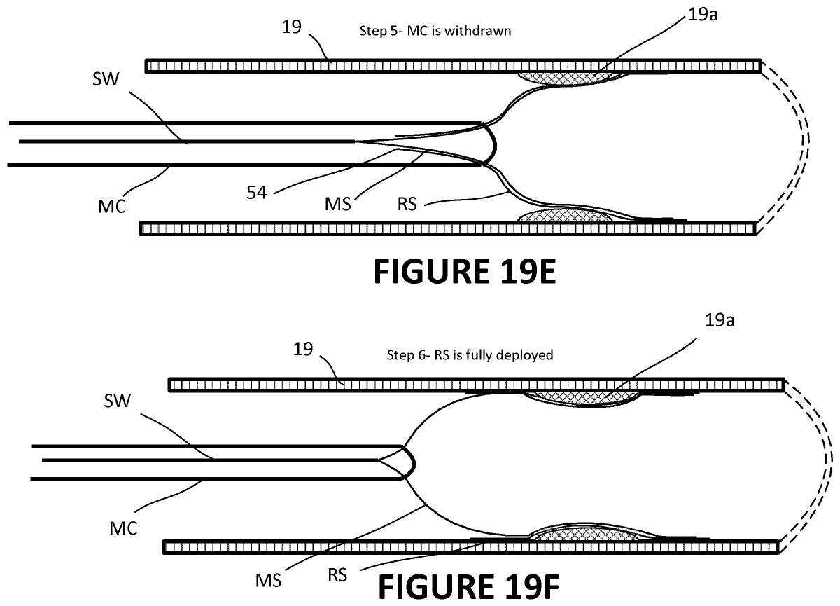

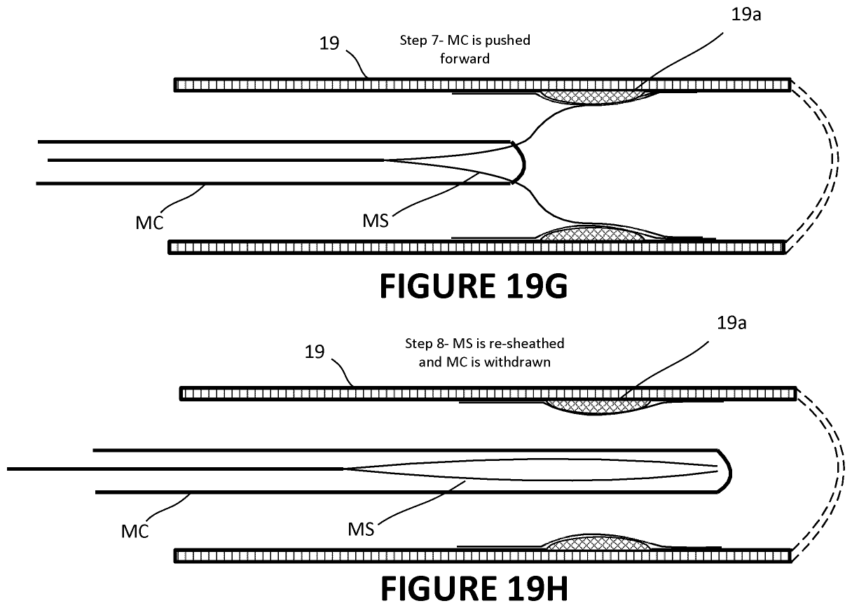

In another embodiment as shown in FIGS. 19A-19H, a co-axial stent is described. In this embodiment, a combined inner metal (MS) and outer resorbable (RS) stent are deployed within a vessel 19 showing a representative lesion 19a. The primary objectives of the co-axial system are:
Improve the deployment of the resorbable stent by using the metal memory/outward spring pressure of the inner metal stent to aid placement of the resorbable stent against the plaque and arterial wall;
Remove the inner metal stent after the outer resorbable stent has been deployed and thus not-limit future treatment options; and,
Enable improved positioning of a non-radio opaque stent (or moderately opaque stent).

In a first embodiment for the placement of a resorbable stent utilizing a co-axial stent, the following steps are undertaken (FIGS. 19A-19H).
a) A microwire (MW) and microcatheter (MC) are advanced to a position past the zone of interest (eg. a plaque) utilizing known procedures and the MW is then removed (Steps 1-3).
b) A co-axial stent system (COSS) is introduced into the proximal end of the MC outside the body and advanced to the distal end of the MC in a compressed state (Step 4). As shown, the COSS includes both an inner metal stent (MS) having a proximal end 50 fixed to a stent wire (SW) at a connection point and an outer resorbable stent (RS) that is frictionally engaged over the MS but is not affixed to either the stent wire or the MS. The RS stent is positioned over the MS such that the distal end of the RS extends a few mm X beyond the distal end of the MS. The proximal end of the RS does not extend proximally beyond the connection point. In other words, the proximal end of the RS is a few mm distal to the connection point 50 as shown by y.
c) When the distal end RSe of the RS is in position, the stent wire is held and the MC is withdrawn such that both the RS and MS are deployed from the distal tip MCe of the MC. As the MC is progressively withdrawn, the RS and MS will expand and engage with the vessel wall 19. Generally, as the MS may have greater spring pressure than the RS, the MS will push against the RS ensuring expansion and engagement of the RS with the vessel wall (Steps 5 and 6). The MS may also be designed to be slightly oversized for the vessel wherein its relaxed state has a diameter greater than the vessel 19.
d) As the MC is continued to be pulled proximally, the proximal end 54 of the RS will exit the MC (Step 6). Continued withdrawal of the MC will deploy more of the MS which will ensure the proximal end of the RS is engaged with the vessel wall.
e) Once the MC has been withdrawn, the COSS will be left in position for a few minutes to allow time for the full expansion of the MS to occur and/or to enable the RS to settle into position.
f) After this time, the MC is advanced distally with the SW being held so that the proximal end of the MC re-sheaths the MS (Step 7). That is, as the MC is pushed distally, the MS will disengage with RS leaving the RS in place while the MS is withdrawn back into the MC (Step 8).
g) When the MS is fully within the MC, the system can be fully withdrawn.

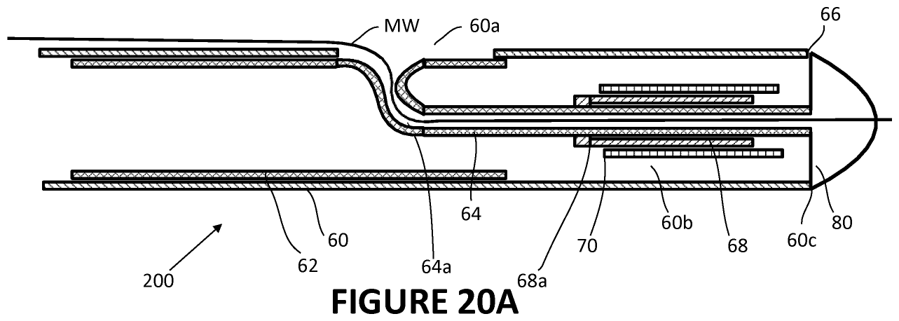

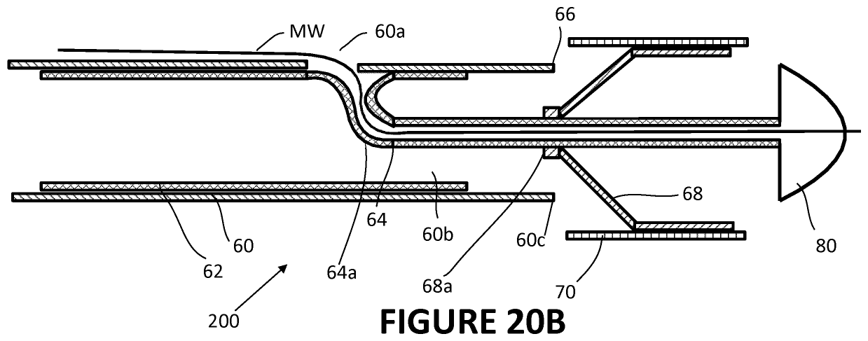

In a separate embodiment, a MC system incorporating a MS is described. As shown in FIGS. 20A and 20B, catheter systems 200 can include catheters where the MW is conveyed to the distal tip of the MC outside of the MC, wherein it passes through the outer wall of the MC into a MC lumen a short distance from the distal tip of the MC.

In accordance with this embodiment, the system 200 includes outer wall catheter 60 and an inner wall catheter 62. The inner wall catheter 62 includes a distal tip inner lumen 64 that defines an inner lumen 64a passageway allowing a MW to passage from outside the system and through the inner lumen to the distal tip 66 of the system. Preferably, an atraumatic tip 80 is attached to the distal tip of the inner wall catheter.

As the inner wall catheter 62 and outer wall catheter 60 are co-axially engaged, the two can move with respect to one another. In order to enable this movement to occur, due to the passage of a MW through the outer wall catheter, the outer wall catheter 60 includes a slot 60a that prevents interference of the MW with the outer wall catheter during co-axial movement.

The inner wall catheter 64 further includes a MS 68 having a proximal end 68a affixed to the outer surface of the distal tip inner lumen 64. The MS is positioned such it is substantially adjacent the distal tip of the system with its distal tip a few mm inside the distal tip as explained in greater detail below. As such, the MS is compressed within the outer wall catheter within the outer wall lumen 60b (not shown to scale). In addition, a RS 70 is compressed within the outer wall lumen 60b outside the MS.

Accordingly, by holding inner wall catheter 62 and pulling the outer wall catheter 60 proximally, the distal end 60c of the outer wall catheter 60 will move proximally relative to the distal tip 64b of the distal tip inner lumen 64. Thus, during this movement, the Rs and MS will project beyond distal end 60c and be able to expand into the vessel (FIG. 20B).

Similarly, by reversing the process, that is holding the inner wall catheter 62 and pushing the outer wall catheter 60 distally, the MS can be made to collapse back into the outer lumen 60b.

As noted, a RS 70 is configured to the outer surface of the MS during manufacturing such that both the MS and RS are collapsed with the outer lumen 60b. Preferably, as noted above, the distal tip of the RS projects slightly distally beyond the MS and the MS projects slightly proximally with respect to the RS.

The RS is thus deployed in a manner described above, with the main difference being that the process of deployment of the RS and MS and re-sheathing of the MS involves manipulation of the inner and outer wall catheters 60 and 62.

The procedures can be applied to both the treatment of unstable plaque and aneurysm.

### 9. Alternate Techniques—Unstable Plaque

9.1. Alternate 1

In another embodiment, the resorbable stent is deployed without complete flow cessation by the BGC and/or MB. In a first alternate technique, the BGC is positioned as described above and a guidewire, microcatheter and stent assembly are advanced past the unstable plaque utilizing the techniques described above.

Preferably, during the advancement of the microwire and microcatheter to beyond the clot, the balloon on the BGC is inflated and active aspiration is conducted during this step to produce transient retrograde flow thus reducing the chance of distal emboli.

The stent assembly is advanced over the guide wire and deployed.

The guide wire is withdrawn through the stent, the BGC is deflated and all equipment is withdrawn.

9.2. Alternate 2

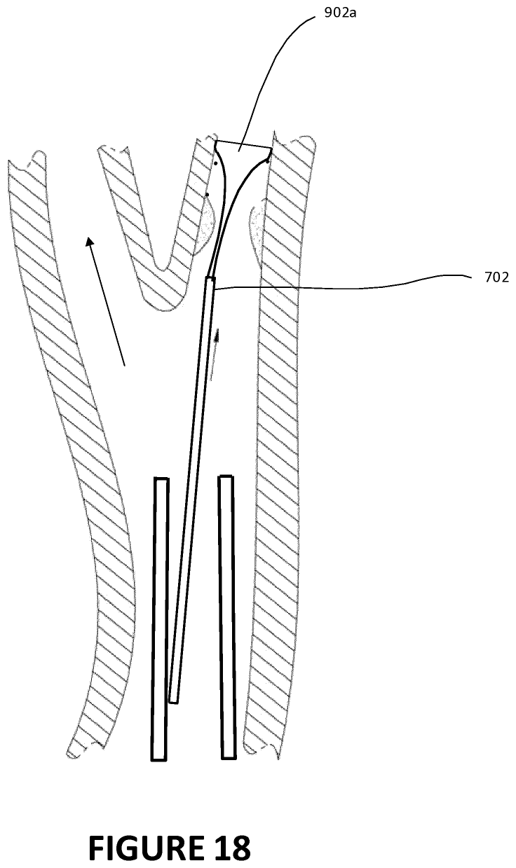

In a second alternate technique, the procedure is conducted without any balloons and hence without flow cessation as shown in FIG. 18. This technique provides an advantage over single or double balloon techniques by reducing the potential for blood pressure fluctuations during the procedure. That is, during a balloon technique, the cessation of blood flow can stimulate the carotid body (carotid glomus) at or adjacent to the CCA bifurcation which can cause significant blood pressure fluctuations during the procedure. As a result of this effect, single or double balloon procedures are generally conducted with an anesthetist to control patient blood pressure as necessary.

Accordingly, procedures conducted without the need of an anesthetist are generally advantaged by speed and cost.

Importantly, if the resorbable stenting is conducted without flow cessation, the resorbable stent can act as distal protection device (DPD) as explained below.

9.3. Distal Protection Devices

As introduced above, current metal stenting procedures of stenosed vessels will usually deploy a distal protection device (DPD) mounted on the guide wire prior to stent deployment. A DPD is typically an inverted basket that can be advanced in a collapsed state past the plaque and deployed by withdrawing a protective sheath. After the DPD is deployed, the metal stent is brought up along the same guide wire and deployed. During this step, the DPD serves to trap any emboli that may be dislodged during stent deployment. After stent deployment, the DPD is collapsed and withdrawn into the its protective sheath.

In the present method and as shown in FIGS. 15 and 18, the use of a DPD would generally not be necessary and thus can save the time used to deploy the DPD as well as the expense of this equipment.

That is, as the resorbable stent of the subject system has a pore size similar to the pore size of a DPD, that is in the range of about 110-250 microns, the act of deploying the resorbable stent will provide the same emboli capturing capabilities of a DPD insomuch as the resorbable stent is self-expanding. In other words, as the resorbable stent deploys distally to the plaque, the distal end will expand against the intima and progressively be deployed in the proximal direction. Thus, any emboli 902b breaking free from the plaque during deployment will be caught between the stent and the intima as shown in FIGS. 15 and 18. Importantly, during this step, the surgeon should ensure that the stent is deployed sufficiently distal to the plaque that the distal tip of the stent is fully contacts the vessel before the stent is deployed across the plaque. This will generally require that the stent is long enough to be deployed in a straight distal section of the ICA.

This technique by virtue of the stent pore size, which is significantly smaller than a typical metal stent will thus retain any emboli between the stent and the intima. Importantly, while the stent is resorbing over time, the emboli will also be resorbed into the intima and/or dissolved as a result of normal blood thinning regimes.

9.4. Treatment of Aneurysm

The treatment of aneurysms using the COSS described above in relation to unstable plaque are similar. Like the placement of a COSS in the CCA/ICA, the COSS can be used as a flow diverter in the treatment of aneurysm utilizing a similar series of steps to deploy the RS and MS and to re-sheath the MS.

Importantly, the COSS can improve the positioning of a RS in that the MS being radio-opaque can provide for accurate positioning of the MS and thus the RS. Depending on the structure of the RS which will generally be constructed of non-radio-opaque materials, the RS can be fabricated with a small amount of metal (eg. tantalum) that could provide some desirable properties to the COSS.

In addition, in some treatment scenarios, it may be desirable to deploy both the RS and MS and leave the MS in place. In this scenario, the RS could be fabricated with a smaller porosity and the MS fabricated with a larger porosity. If both are left in place after deployment, the RS will ensure that the aneurysm stabilizes over a period of time by fully occluding blood flow into and around the edges of the aneurysm thus providing the appropriate period of time for the aneurysm to heal. However, as the RS will resorb over a period of time, the tight porosity of the RS will disappear, and the larger porosity of the MS will remain. As a result, while metal may remain, the porosity of the MS may still permit access to the aneurysm at a time in the future through the pores of the MS thus making available some additional treatment options available should access to the aneurysm be required. This is different than treatment with a tight MS as deployment of a single MS will generally utilize a MS having small pores that prevent blood flow through them.

If a COSS is designed where both the RS and MS are deployed, the RS/MS may be conveyed and deployed through a MC as described above and the MS detached from the stent wire utilizing known detachment techniques.

9.5. Equivalents

At least the following equivalents and scope are contemplated.

An example location for the unstable plaque 404 is described with respect to FIGS. 8 to 18. However, this location is merely exemplary. An unstable plaque may be located in the CCA 400a or the ICA 400b or a combination thereof. The geometry of the resorbable stent would be readily apparent to the skilled person in view of the discussion provided herein.

FIGS. 8 to 17 contemplate balloon deployment in each of the CCA and the ECA to substantially arrest blood flow at an unstable plaque. It will be appreciated that occlusion of at least any two of the three arteries proximal to the CCA bifurcation could substantially arrest blood flow at the unstable plaque.

If one or more balloons are used to substantially arrest blood flow at an unstable plaque, it will be appreciated that the balloons may be deflated either by manual input by someone operating the BGC or may automatically deflate after a predetermined period of time. In a further embodiment, the distal balloons may be a self deflating detachable balloon that may be detached into the ECA.

In another embodiment, although not required, a microcatheter does not need to be advanced along a guidewire, and instead a resorbable stent may be advanced directly along the guide wire. In a further embodiment the guide wire may not be necessary if adequate control of the resorbable stent can be effected without the guidewire or the microcatheter.

9.6. Uses and Kits

In addition to the methods described above, uses of a resorbable stent and kits are also contemplated. The uses and kits described below encompass at least features described in the methods disclosed above and its equivalents.

A use of a resorbable stent is contemplated to stabilize an unstable plaque in a patient for a therapeutically effective time period at a bifurcation of a CCA into an ICA and an ECA, where the resorbable stent is deployed under substantial arrest of blood flow at the unstable plaque. A use of a RS as a flow diverter for the treatment of aneurysm is also contemplated.

A use of a co-axial resorbable and metal stent is contemplated. Specifically, the use may be of a COSS to stabilize an unstable plaque in a patient for a therapeutically effective time period at a bifurcation of a CCA into an ICA and an ECA, where the resorbable stent is deployed under substantial arrest of blood flow at the unstable plaque. A use of a COSS as a flow diverter for the treatment of aneurysm is also contemplated.

A kit for the treatment of an unstable plaque and as flow diverter for the treatment of aneurysm in a patient is also contemplated. Kits may include one or more devices, the one or more devices adapted to substantially arrest blood flow at the unstable plaque adjacent to a bifurcation of a CCA into an ICA and an ECA or as a flow diverter for the treatment of aneurysm. The kit may further include or merely comprise at least one COSS having a resorbable stent adapted to stabilize the unstable plaque/aneurysm for a therapeutically effective time period.

Kits may comprise within individual or separate packing a combination one or more of a first BGC, a second BGC that is deployable through the first GBC, one or more guide wires, one or more microcatheters and one or more stent assemblies having one or more resorbable stents as well as re-sheathable metal stents. The resorbable stents may be provided with a variety of features that allow a surgeon to select desired functional and structural characteristics for a specific case.

For example, metal and resorbable stents may combinations of the following functional/structural characteristics including a range of:
diameters;
lengths;
tapers;
compressive stiffnesses;
pore sizes;
drug coatings; and,
resorb times.

Generally, a RS stent will be designed to resorb at a rate proportional to blood flow. Hence, to the extent that a RS protrudes into a blood vessel or covers a blood vessel, the RS will begin to resorb/erode at positions having the highest blood flow rates and progress to areas having lower blood flow rates.

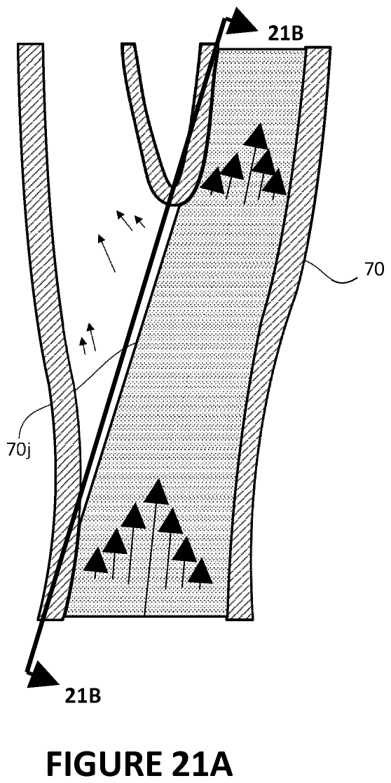

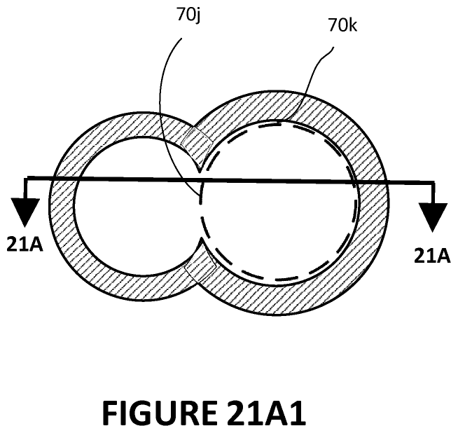

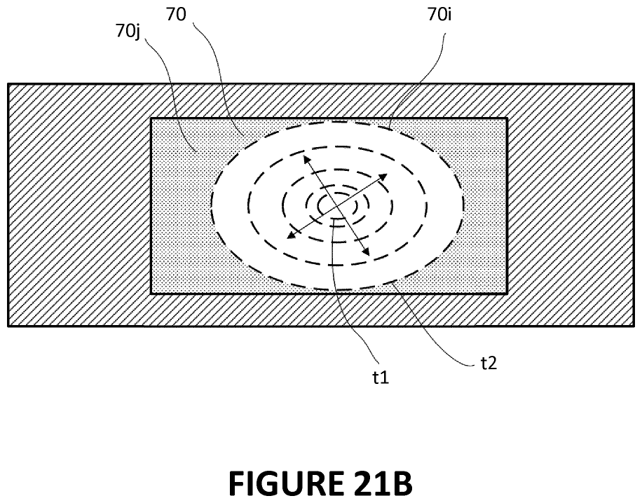

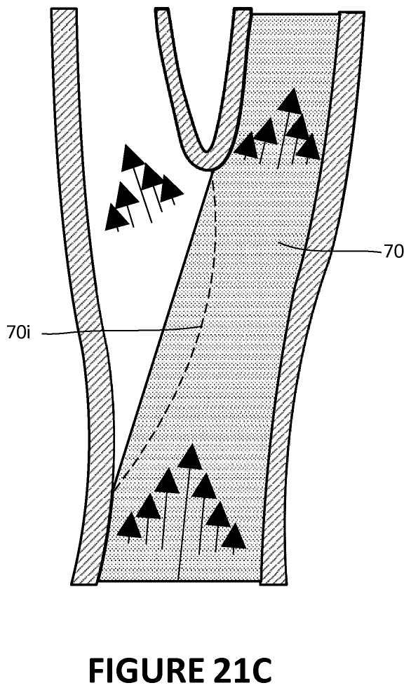

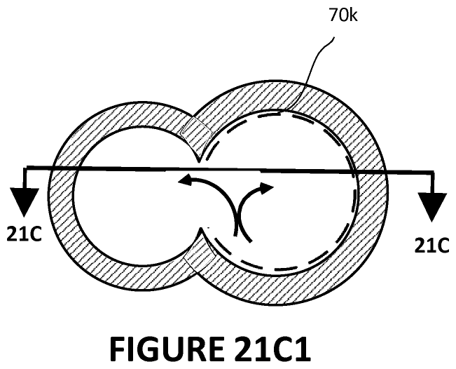

With reference to FIGS. 21A, 21A1, 21B, 21C and 21C1 which are various schematic cross-sections of a vessel with a deployed RS (eg. a CCA/ICA/ECA bifurcation), the process by which a RS is resorbed is shown. That is, as shown in FIGS. 21A and 21A1, a RS 70 may be deployed such that it partially extends into/over another vessel wherein edges/surfaces 70j of the RS will not be engaged with the vessel wall. In this example, at deployment, the RS occludes one vessel such that blood flow into the occluded vessel is low and only occurs through pores of the RS as shown schematically by the flow arrows in each vessel segment.

Over time, the edges of the RS that are not engaged with the vessel wall 70j and exposed to the greatest flow, will begin to erode/resorb as shown by dotted lines 70i in FIG. 21B thus forming a hole through the RS and allowing increased blood flow into the occluded vessel as shown in FIGS. 21C and 21C1. Typically, the pattern of erosion will be a progressively larger ellipse as shown by ellipses t1 and t2 which represent the size of the elliptical hole at two times.

Those surfaces 70k that are in contact with the vessel wall may depending on a number of factors (including the design of the rate of resorption) either become endotheliazied and thus not resorb or may partially or completely resorb.

The size of the RS pores may also affect the rate of resorption as the rate of flow through the pores may be variable.

Importantly, the integrity of the stent contacting the vessel wall will be maintained during the endotheliazation process and/or during resorption of RS in that resorption will generally progress from an exposed edge of the RS away from a vessel wall towards the vessel wall. Thus, when a resorbed edge reaches the vessel wall, resorption may cease in the case where the RS has become endothelialized or may continue at a lower rate as blood flow rate at the wall may be slower. Also, in the case where a vessel is partially obstructed, blood flow rate reduction would preferably only occur for a short period of time and full flow may be re-established within a few days/weeks. This may provide a further advantage of reducing the need for anti-platelet medication in the patient.

Various stents may have different combinations of each of the above structures and functionalities.

### 10. Stent Design

As noted, RS and MS may have a plurality of features that make it suitable for use in treating unstable plaque or aneurysm. Given the variability in the size and location of plaque being treated adjacent the CCA bifurcation, stents having different lengths and features may be utilized. Similarly, given the variability of the size and structure of aneurysms, MS and RS having different lengths and features may be utilized.

For example, a plaque in the ICA may be 7-9 mm in length and extend into the ICA 0.5-1 mm. The center of the plaque may be 4-6 mm from the bifurcation. Generally, in order to enable the stent to be useful as a DPD, the stent would typically be longer than a stent that is used with a separate DPD.

That is, as shown in FIG. 18, as the stent must contact the intima before it is fully effective as a DPD, and there is a distance between the distal tip of the stent and the distal tip of the deployment catheter before the distal tip of the stent is fully engaged with the intima, the surgeon will typically need to deploy the stent a few mm further in the distal direction to enable this. Hence, in comparison to current stents used at this location, the stent in accordance with the invention will typically be a few mm longer. Moreover, particularly when the procedure is conducted without flow cessation, the initial step of stent deployment should be conducted further in the distal direction to minimize contact with the plaque and the risk of disturbing it. As such, a stent will typically be 30-50 mm long and more specifically 40-42 mm long.

The ultimate selection of the length and other features of the stent will be determined by the surgeon having regard to the particular characteristics of the plaque/aneurysm.

It should also be noted that braided metal stents having the above structural features could be developed and utilized. In particular, these stents could also be effective as DPDs as described above. In a COSS system, the RS may also function as a DPD.

Further still, in the design of a COSS system for aneurysms, as noted above, one design contemplates a RS having smaller pore sizes and a MS having larger pore sizes such that when the RS has resorbed the larger pore sizes of the MS may enable access into the aneurysm at a later time. In another embodiment, the RS is a mesh of very fine wires with a defined pore size that are solution cast with a RS material so as to partially fill in the pores of the MS. Thus, in this embodiment, the RS component and MS component are overlaid with respect to one another such that the effective pore size of the MS increases over time as resorbable material is eroded away from the MS material, thus enabling future access through the MS to gain access to an aneurysm if and when necessary.

In another embodiment, the RS is seated by positioning and inflation of a balloon after the RS has been deployed. In this case, the radio-opaque markers on the balloon can provide positioning information to the physician when deploying the RS.

### CONCLUSION

While this invention has been particularly shown and described with references to embodiments thereof, it will be understood by those skilled in the art that various changes in form and details may be made therein without departing from the scope of the invention encompassed by the appended claims.

1. (canceled)

2. (canceled)

3. (canceled)

4. (canceled)

5. (canceled)

6. (canceled)

7. (canceled)

8. (canceled)

9. (canceled)

10. (canceled)

11. (canceled)

12. (canceled)

13. (canceled)

14. (canceled)

15. (canceled)

16. (canceled)

17. (canceled)

18. A kit for the treatment of an unstable plaque or an aneurysm at a desired location in a patient, the kit comprising:
at least one guide catheter (GC) adapted for placement proximal to the desired location;
at least one guide wire adapted for placement distal to the desired location;
at least one microcatheter adapted for placement distal to the desired location over the guide wire;
at least one resorbable stent (RS) assembly adapted for placement adjacent to the desired location and deployable through the at least one microcatheter each RS assembly having a RS adapted to stabilize the unstable plaque or aneurysm for a therapeutically effective time period and resorbable into the patient over a resorb time.

19. The kit as in claim 18 where the GC is at least one balloon guide catheter (BGC) for occluding blood flow.

20. The kit as in claim 18 further comprising at least one micro-balloon (MB) for occluding blood flow through the ECA.

21. The kit as in claim 18 where the kit includes at least two resorbable stent assemblies each having a resorbable stent, and where the resorbable stents have at least one different structural and/or functional property from each other, selected from any one of or a combination of stent diameter, stent length, stent taper, stent compressive stiffness, stent pore size; stent drug coating and stent resorb time.

22. A resorbable stent (RS) for deployment within an arterial vessel of a patient at a desired location, the RS comprising:
a cylindrical body having a plurality of pore openings in the range of 110-250 microns diameter and a void space of greater than 50% of the cylindrical body, the cylindrical body collapsible within a microcatheter and deployable from the microcatheter for placement with the arterial vessel at the desired location and wherein the cylindrical body is self-expanding upon deployment within an artery and resorbable into the patient after deployment.

23. The resorbable stent as in claim 22 wherein the stent has a resorb time of one week or less.

24. The resorbable stent as in claim 22 wherein the stent has a resorb time of one month or less.

25. The resorbable stent as in claim 22 wherein the stent has a resorb time of two months or less.

26. The resorbable stent according to claim 22 wherein the stent is poly lactic-co-glycolic acid.

27. The resorbable stent as in claim 22 wherein the cylindrical body is a weave of poly lactic-co-glycolic acid fibers, the fibers having a diameter in the range of 30-50 microns.

28. The resorbable stent according to claim 22 wherein the cylindrical body has an overall length of 3-5 cm.

29. The resorbable stent according to claim 22 wherein the cylindrical body of the RS has a rate of resorption proportional to blood flow through the stent tines and wherein regions of the stent subjected to higher blood flow will resorb faster than regions of the stent having lower blood flow.

30. The resorbable stent as in claim 29 wherein the cylindrical body has resorb properties wherein the cylindrical body resorbs progressively along exposed edges of the cylindrical body not in contact with a vessel wall towards a vessel wall so as to maintain a structural integrity of the cylindrical body during resorption.

31. The resorbable stent as in claim 30 wherein the cylindrical body has resorb properties such that during resorption of exposed edges of the cylindrical body not in contact with a vessel wall, surfaces of the cylindrical body in contact with a vessel wall endothelialize and do not resorb.

32. A co-axial stent system (COSS) comprising:
a catheter system retaining:
a collapsible resorbable stent (RS) having:
a collapsible cylindrical body for compressed containment within the catheter system; and,
resorption properties where the RS is resorbable within a patient over a resorb time;
a collapsible metal stent (MS) affixed to a stent wire (SW) passing through the catheter system, the MS having:
a collapsible cylindrical body for compressed containment within the catheter system and the RS;
sufficient self-expansion properties enabling the MS to bias the RS against the arterial vessel upon deployment;
wherein the MS may be unsheathed and re-sheathed from the catheter system and wherein upon deployment of the RS and re-sheathing of the MS, the RS remains deployed within an arterial vessel.

33. A co-axial stent system (COSS) comprising:
a catheter system having an outer catheter, an inner catheter and a microwire having a proximal zone running external to the outer catheter and a distal zone entering the inner catheter through a distal slot in the outer catheter, the inner catheter having a distal inner zone retaining:
a collapsible resorbable stent (RS) having:
a collapsible cylindrical body for compressed containment within the distal inner zone of the inner catheter between the outer catheter and inner catheter; and,
resorption properties where the RS is resorbable within a patient over a resorb time;
a collapsible metal stent (MS) affixed to the distal inner zone between the outer catheter and the inner catheter, the MS having:
a collapsible cylindrical body for compressed containment within the distal inner zone and within the RS;
sufficient self-expansion properties enabling the MS to bias the RS against the arterial vessel upon deployment;
wherein the MS may be unsheathed and re-sheathed from the catheter system and wherein upon deployment of the RS and re-sheathing of the MS, the RS remains deployed within an arterial vessel.

34. (canceled)

35. (canceled)

36. (canceled)

37. (canceled)

38. (canceled)

39. (canceled)

40. (canceled)

41. (canceled)

42. (canceled)

43. (canceled)

44. (canceled)

45. (canceled)

46. (canceled)

47. (canceled)

48. (canceled)

49. (canceled)
